(19) United States
(12) Patent Application Publication
(10) Pub. No.: US 2009/0130232 A1
(43) Pub. Date: May 21, 2009

(54) Composition and method for treatment of oral inflammation an ulceration

(76) Inventor: Mohammed Zahra, Norfolk, NE (US)

Correspondence Address: SUITER SWANTZ PC LLO, 14301 FNB PARKWAY, SUITE 220, OMAHA, NE, 68154, US

(21) Appl. No.: 11/986,129

(22) Filed: Nov. 20, 2007

(51) Int. Cl. A61K 31/135; A61K 31/167; A61K 31/225; A61K 31/573; A61K 31/7048; A61K 33/08; A61P 43/00

(52) U.S. Cl. 424/690; 514/171; 514/31; 514/548; 514/626; 514/648

(57) ABSTRACT

The present invention is a composition and method for treating oral lesions.

A composition for treatment of oral lesions may comprise: (a) an anesthetic composition; (b) an antifungal composition; (c) a steroid composition; (d) an antihistamine composition; and (e) an antacid composition, wherein the therapeutic composition is substantially free of alcohol.

A method for treatment may comprise: (a) administering to a patient an effective amount of a composition, the therapeutic composition comprising: (i) an anesthetic composition; (ii) an antifungal composition; (iii) a steroid composition; (iv) an antihistamine composition; and (v) an antacid composition, wherein the therapeutic composition is substantially free of alcohol.

A method of producing a composition may comprise: combining (a) an anesthetic composition, (b) an antifungal composition, (c) a steroid composition, (d) an antihistamine composition, and (e) an antacid composition, wherein the therapeutic composition is substantially free of alcohol.

### BACKGROUND

Aggressive cancer treatment may have toxic effects on normal cells as well as cancer cells. Patients with cancer of the head and neck who are undergoing radiation therapy, chemotherapy or a combination of both modalities may suffer adverse affects due to the treatments. The gastrointestinal tract, including the mouth, is especially sensitive to such treatments. As such, patients may develop painful mouth sores such as Stomatitis or inflammations of mucosal membranes, such as Mucositis.

Such conditions can contribute to oral infections, inability to taste normally and pain arising from the resulting open sores that can develop. Mucositis can become so painful that the patient will not eat or drink, contributing to dehydration and malnutrition. Also, radiation therapy to the head and neck for cancers in those areas commonly injure saliva glands and the inside of the mouth which can cause dry mouth, leading to dental disease.

Such oral problems may make it difficult for the cancer patient to receive a complete dose of chemotherapy or radiation therapy. Sometimes treatment must be stopped completely.

It should be noted that Mucositis and Stomatitis problems are not restricted to cancer patients, as they frequently occur in HIV patients, particularly when associated with Kaposi's sarcoma, in patients affected with non-Hodgkin's lymphoma, in debilitated elderly patients and in patients receiving BRM treatments.

Therefore, it would be desirable to provide a treatment for the relief of the symptoms related to Mucositis, Stomatitis, and other oral conditions.

### SUMMARY

Accordingly, the present disclosures are directed to a composition and method for the treatment of oral lesions.

A composition for treatment of oral lesions may comprise: (a) an anesthetic composition; (b) an antifungal composition; (c) a steroid composition; (d) an antihistamine composition; and (e) an antacid composition, wherein the therapeutic composition is substantially free of alcohol.

A method for treatment of oral lesions may comprise: (a) administering to a patient an effective amount of a composition, the therapeutic composition comprising: (i) an anesthetic composition; (ii) an antifungal composition; (iii) a steroid composition; (iv) an antihistamine composition; and (v) an antacid composition, wherein the therapeutic composition is substantially free of alcohol.

It is to be understood that both the foregoing general description and the following detailed description are exemplary and explanatory only and are not restrictive of the invention claimed. The accompanying drawings, which are incorporated in and constitute a part of the specification, illustrate an embodiment of the invention and together with the general description, serve to explain the principles of the invention.

### DETAILED DESCRIPTION OF THE INVENTION

The following discussion is presented to enable a person skilled in the art to make and use the present teachings. Various modifications to the illustrated embodiments will be readily apparent to those skilled in the art, and the generic principles herein may be applied to other embodiments and applications without departing from the present teachings. Thus, the present teachings are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the present teachings. Skilled artisans will recognize the examples provided herein have many useful alternatives and fall within the scope of the present teachings.

The oral mucosa is composed of rapidly dividing cells which are highly sensitive to the effects of radiation and chemotherapy. During such treatments, patients may develop oral inflammation and/or ulcerations due to conditions such as Mucositis and Stomatitis. As such, patients may have difficulty consuming and swallowing food due to the oral ulcerations, thereby significantly diminishing their quality of life.

Topical administration of therapeutic compositions, such as those described herein, may reduce the pain associated with such ulcerations.

A therapeutic composition for treatment may comprise: an anesthetic composition; an antifungal composition; a steroid composition; an antihistamine composition; and an antacid composition.

The therapeutic composition may be substantially alcohol-free so as to avoid unnecessary irritation of the inflamed or ulcerated region due to the therapeutic composition.

The anesthetic composition may comprise an amino ester or an amino amide. The anesthetic composition may be selected from the group comprising: lidocaine, ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, pharmaceutically acceptable salts thereof, and mixtures thereof. Particularly, the anesthetic composition may be a 2% lidocaine solution. The anesthetic composition may be present in an amount of from about 20-30% by volume of the therapeutic composition.

The antifungal composition may be a polyene antifungal composition. The antifungal may be selected from the group consisting of: nystatin, amphotericin A, amphotericin B, aurofacin, candicidin, candidin, filipin, levorin, mycoheptin, perimycin, pimaricin, polyfungin, rimocidin, trichomycin, pharmaceutically acceptable salts thereof, and mixtures thereof. Particularly, the antifungal composition may be a 100,000 unit/ml nystatin solution. The antifungal composition may be present in an amount of from about 20-30% by volume of the therapeutic composition.

The steroid composition may comprise a corticosteroid composition. The steroid composition may be selected from the group consisting of: prednisone, algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methyiprednisolone, methyiprednisolone acetate, methyiprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21 (beta-D-glucuronide), prednylidene, procinonide, tralonide, triamcinolone, triamcinolone acetonide, triamcinolone acetonide 21-palmitate, triamcinolone diacetate, triamcinolone hexacetonide, wortmannin, pharmaceutically acceptable salts thereof, and mixtures thereof.

The steroid composition may be a solid or powdered formulation of prednisone. The steroid composition may be present in an amount of from about 0.15 to 0.35 mg per ml of the therapeutic composition.

The antihistamine composition may be selected from the group consisting of: diphenhydramine, astemizole, azatadine, azelastine, acrivastine, brompheniramine, chlorpheniramine, clemastine, cyclizine, carebastine, cyproheptadine, carbinoxamine, descarboethoxyloratadine, doxylamine, cetirizine, dimenhydrinate, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine, triprolidine, pharmaceutically acceptable salts thereof, and mixtures thereof.

The antihistamine composition may be a 2.5 mg/ml diphenhydramine solution, such as that marketed under the brand name Benadryl®. The antihistamine composition may be present in an amount of from about 20-30% by volume of the therapeutic composition.

The antacid composition may be selected from the group consisting of: a composition comprising: aluminum hydroxide, magnesium hydroxide, aluminum carbonate, aluminum phosphate, aluminum hydroxy carbonate, aluminum citrate, dihydroxyaluminum sodium carbonate, aluminum magnesium glycinate, dihydroxyaluminum aminoacetate, dihydroxyaluminum aminoacetic acid, bismuth aluminate, bismuth carbonate, bismuth subcarbonate, bismuth subgallate, bismuth subnitrate, calcium carbonate, calcium hydroxide, calcium phosphate, calcium citrate, calcium citrate malate, hydrated magnesium aluminate, activated sulfate, magnesium aluminate, magnesium aluminosilicates, magnesium carbonate, magnesium glycinate, magnesium oxide, magnesium trisilicate, potassium carbonate, potassium phosphate, potassium citrate, sodium carbonate, sodium bicarbonate, sodium phosphate, sodium citrate, pharmaceutically acceptable salts thereof, and mixtures thereof.

The antacid composition may be a solution having 30-90 g of aluminum hydroxide and 30-90 g of magnesium hydroxide per ml of solution, such as that marketed under the brand name Maalox®. The antacid composition may be present in an amount of from about 20-30% by volume of the therapeutic composition.

The therapeutic composition may further comprise a flavoring. The therapeutic composition may be substantially free of antibiotics.

The therapeutic composition may further comprise a composition selected from the group consisting of: acetaminophen and acetylsalicylic acid. The acetaminophen or acetylsalicylic acid may be present in the therapeutic composition in an amount of from about 1.00 to 1.70 mg per ml of therapeutic solution. The acetaminophen or acetylsalicylic acid may be present in the therapeutic composition in an amount of about 1.35 mg per ml of therapeutic solution.

A method of treatment may comprise: administering to a patient an effective amount of a therapeutic composition, the therapeutic composition comprising: an anesthetic composition; an antifungal composition; a steroid composition; an antihistamine composition; and an antacid composition, wherein the therapeutic composition is substantially free of alcohol.

The therapeutic composition may be administered to a patients oral cavity. The method of treatment of claim may further comprise administering the therapeutic composition at least two times daily. The therapeutic composition may be administered in a volume of about 10 ml per treatment.

The administered therapeutic composition may comprise: an anesthetic composition; an antifungal composition; a steroid composition; an antihistamine composition; and an antacid composition.

The administered therapeutic composition may be substantially alcohol-free so as to avoid unnecessary irritation of the inflamed or ulcerated region due to the administered therapeutic composition.

The anesthetic composition may comprise an amino ester or an amino amide. The anesthetic composition may be selected from the group comprising: ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, pharmaceutically acceptable salts thereof, and mixtures thereof. Particularly, the anesthetic composition may be a 2% lidocaine solution. The anesthetic composition may be present in an amount of from about 20-30% by volume of the administered therapeutic composition.

The antifungal composition may be a polyene antifungal composition. The antifungal may be selected from the group consisting of: nystatin, amphotericin A, amphotericin B, aurofacin, candicidin, candidin, filipin, levorin, mycoheptin, perimycin, pimaricin, polyfungin, rimocidin, trichomycin, pharmaceutically acceptable salts thereof, and mixtures thereof. Particularly, the antifungal composition may be a 100,000 unit/ml nystatin solution. The antifungal composition may be present in an amount of from about 20-30% by volume of the administered therapeutic composition.

The steroid composition may comprise a corticosteroid composition. The steroid composition may be selected from the group consisting of: prednisone, algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methyiprednisolone, methyiprednisolone acetate, methyiprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21 (beta-D-glucuronide), prednylidene, procinonide, tralonide, triamcinolone, triamcinolone acetonide, triamcinolone acetonide 21-palmitate, triamcinolone diacetate, triamcinolone hexacetonide, wortmannin, pharmaceutically acceptable salts thereof, and mixtures thereof.

The steroid may be a solid or powdered formulation of prednisone. The steroid composition may be present in an amount of from about 0.15 to 0.35 mg per ml of the administered therapeutic composition.

The antihistamine composition may be selected from the group consisting of: diphenhydramine, astemizole, azatadine, azelastine, acrivastine, brompheniramine, chlorpheniramine, clemastine, cyclizine, carebastine, cyproheptadine, carbinoxamine, descarboethoxyloratadine, doxylamine, cetirizine, dimenhydrinate, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine, triprolidine, pharmaceutically acceptable salts thereof, and mixtures thereof.

The antihistamine composition may be a 2.5 mg/ml diphenhydramine solution, such as that marketed under the brand name Benadryl®. The antihistamine composition may be present in an amount of from about 20-30% by volume of the administered therapeutic composition.

The antacid composition may be selected from the group consisting of: a composition comprising: aluminum hydroxide, magnesium hydroxide, aluminum carbonate, aluminum phosphate, aluminum hydroxy carbonate, aluminum citrate, dihydroxyaluminum sodium carbonate, aluminum magnesium glycinate, dihydroxyaluminum aminoacetate, dihydroxyaluminum aminoacetic acid, bismuth aluminate, bismuth carbonate, bismuth subcarbonate, bismuth subgallate, bismuth subnitrate, calcium carbonate, calcium hydroxide, calcium phosphate, calcium citrate, calcium citrate malate, hydrated magnesium aluminate, activated sulfate, magnesium aluminate, magnesium aluminosilicates, magnesium carbonate, magnesium glycinate, magnesium oxide, magnesium trisilicate, potassium carbonate, potassium phosphate, potassium citrate, sodium carbonate, sodium bicarbonate, sodium phosphate, sodium citrate, pharmaceutically acceptable salts thereof, and mixtures thereof.

The antacid composition may be a solution having 30-90 g of aluminum hydroxide and 30-90 g of magnesium hydroxide per ml of solution, such as that marketed under the brand name Maalox®. The antacid composition may be present in an amount of from about 20-30% by volume of the administered therapeutic composition.

The administered therapeutic composition may further comprise a flavoring. The therapeutic composition may be substantially free of antibiotics.

The administered therapeutic composition may further comprise a composition selected from the group consisting of: acetaminophen and acetylsalicylic acid. The acetaminophen or acetylsalicylic acid may be present in the therapeutic composition in an amount of from about 1.00 to 1.70 mg per ml of administered therapeutic solution. The acetaminophen or acetylsalicylic acid may be present in the therapeutic composition in an amount of about 1.35 mg per ml of administered therapeutic solution.

It is believed that the present invention and many of its attendant advantages will be understood from the foregoing description, and it will be apparent that various changes may be made in the form, construction, and arrangement of the components thereof without departing from the scope and spirit of the invention or without sacrificing all of its material advantages. The form herein before described being merely an explanatory embodiment thereof, it is the intention of the following claims to encompass and include such changes.

What is claimed is:

1. A composition comprising:
an anesthetic composition;
an antifungal composition;
a steroid composition;
an antihistamine composition; and
an antacid composition,
wherein the therapeutic composition is substantially free of alcohol.

2. The therapeutic composition of claim 1, wherein the anesthetic composition is selected from the group consisting of:
lidocaine, ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, euprocin, fenalcomine, formocaine, hexylcaine, hydroxyteteracaine, isobutyl p-aminobenzoate, leucinocaine, levoxadrol, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, pharmaceutically acceptable salts thereof, and mixtures thereof.

3. The therapeutic composition of claim 2, wherein the anesthetic composition is lidocaine.

4. The therapeutic composition of claim 1, wherein the antifungal composition is selected from the group consisting of:
nystatin, amphotericin A, amphotericin B, aurofacin, candicidin, candidin, filipin, levorin, mycoheptin, perimycin, pimaricin, polyfungin, rimocidin, trichomycin, pharmaceutically acceptable salts thereof, and mixtures thereof.

5. The therapeutic composition of claim 4, wherein the antifungal composition is nystatin.

6. The therapeutic composition of claim 1, wherein the steroid composition is selected from the group consisting of:
prednisone, algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methyiprednisolone, methyiprednisolone acetate, methyiprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21 (beta-D-glucuronide), prednylidene, procinonide, tralonide, triamcinolone, triamcinolone acetonide, triamcinolone acetonide 21-palmitate, triamcinolone diacetate, triamcinolone hexacetonide, wortmannin, pharmaceutically acceptable salts thereof, and mixtures thereof.

7. The therapeutic composition of claim 6, wherein the steroid composition is prednisone.

8. The therapeutic composition of claim 1, wherein the antihistamine composition is selected from the group consisting of:
diphenhydramine, astemizole, azatadine, azelastine, acrivastine, brompheniramine, chlorpheniramine, clemastine, cyclizine, carebastine, cyproheptadine, carbinoxamine, descarboethoxyloratadine, doxylamine, cetirizine, dimenhydrinate, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine, triprolidine, pharmaceutically acceptable salts thereof, and mixtures thereof.

9. The therapeutic composition of claim 8, wherein the antihistamine composition is diphenhydramine.

10. The therapeutic composition of claim 1, wherein the antacid composition is selected from the group consisting of:
a composition comprising aluminum hydroxide and magnesium hydroxide, aluminum hydroxide, magnesium hydroxide, aluminum carbonate, aluminum phosphate, aluminum hydroxy carbonate, aluminum citrate, dihydroxyaluminum sodium carbonate, aluminum magnesium glycinate, dihydroxyaluminum aminoacetate, dihydroxyaluminum aminoacetic acid, bismuth aluminate, bismuth carbonate, bismuth subcarbonate, bismuth subgallate, bismuth subnitrate, calcium carbonate, calcium hydroxide, calcium phosphate, calcium citrate, calcium citrate malate, hydrated magnesium aluminate, activated sulfate, magnesium aluminate, magnesium aluminosilicates, magnesium carbonate, magnesium glycinate, magnesium oxide, magnesium trisilicate, potassium carbonate, potassium phosphate, potassium citrate, sodium carbonate, sodium bicarbonate, sodium phosphate, sodium citrate, pharmaceutically acceptable salts thereof, and mixtures thereof.

11. The therapeutic composition of claim 10, wherein the antacid composition is a composition comprising: aluminum hydroxide and magnesium hydroxide

12. The therapeutic composition of claim 1, further comprising:
a composition selected from the group consisting of: acetaminophen and acetylsalicylic acid.

13. The therapeutic composition of claim 1,
wherein the anesthetic composition is present in an amount of from about 20-30% by volume of the therapeutic composition;
wherein the antifungal composition is present in an amount of from about 20-30% by volume of the therapeutic composition;
wherein the antihistamine composition is present in an amount of from about 20-30% by volume of the therapeutic composition;
wherein the antacid composition is present in an amount of from about 20-30% by volume of the therapeutic composition; and
wherein the steroid composition is present in an amount of 0.15 to 0.35 mg per ml of the therapeutic composition.

14. The therapeutic composition of claim 13, wherein the anesthetic composition comprises:
a 2% lidocaine solution.

15. The therapeutic composition of claim 13, wherein the antifungal composition comprises:
a 100,000 unit/ml nystatin solution.

16. The therapeutic composition of claim 13, wherein the antihistamine composition comprises:
a 2.5 mg/ml diphenhydramine solution.

17. The therapeutic composition of claim 13, wherein the antacid composition comprises:
a solution having 30-90 g of aluminum hydroxide and 30-90 g of magnesium hydroxide per ml of solution.

18. The therapeutic composition of claim 13, further comprising:
a composition selected from: acetaminophen or acetylsalicylic acid wherein the acetaminophen or acetylsalicylic acid is present in an amount of from
about 1.00 to 1.70 mg per ml of therapeutic solution.

19. The therapeutic composition of claim 1, wherein the therapeutic composition is substantially free of antibiotics.

20. The therapeutic composition of claim 1, further comprising:
a flavoring.

21. A method of treatment comprising:
administering to a patient an effective amount of a therapeutic composition, the therapeutic composition comprising:
an anesthetic composition;
an antifungal composition;
a steroid composition;
an antihistamine composition; and
an antacid composition,
wherein the therapeutic composition is substantially free of alcohol.

22. The method of treatment of claim 21, further comprising,
administering the therapeutic composition at least two times daily.

23. The method of treatment of claim 21, wherein administering to a patient an effective amount of a composition further comprises:
administering an effective amount of a composition to an oral cavity of the patient.

24. The method of treatment of claim 21,
wherein the anesthetic composition is present in an amount of from about 20-30% by volume of the therapeutic composition;
wherein the antifungal composition is present in an amount of from about 20-30% by volume of the therapeutic composition;
wherein the antihistamine composition is present in an amount of from about 20-30% by volume of the therapeutic composition;
wherein the antacid composition is present in an amount of from about 20-30% by volume of the therapeutic composition; and
wherein the steroid composition is present in an amount of from about 0.15 to 0.35 mg per ml of the therapeutic composition.

25. The method of treatment of claim 21,
wherein the effective amount of the therapeutic composition is 10 ml.

26. A method of producing a composition, the method comprising:
combining an anesthetic composition, an antifungal composition, a steroid composition, an antihistamine composition, and an antacid composition,
wherein the therapeutic composition is substantially free of alcohol.

27. The method of producing a composition of claim 26,
wherein the anesthetic composition is present in an amount of from about 20-30% by volume of the therapeutic composition;
wherein the antifungal composition is present in an amount of from about 20-30% by volume of the therapeutic composition;
wherein the antihistamine composition is present in an amount of from about 20-30% by volume of the therapeutic composition;
wherein the antacid composition is present in an amount of from about 20-30% by volume of the therapeutic composition; and
wherein the steroid composition is present in an amount of from about 0.15 to 0.35 mg per ml of the therapeutic composition.
